(19) United States
(12) Patent Application Publication
(10) Pub. No.: US 2025/0207205 A1
(43) Pub. Date: Jun. 26, 2025

(54) REAGENT, KIT, AND METHOD FOR DETECTING PIG ECONOMIC TRAIT-ASSOCIATED MUTATION SITE

(71) Applicant: Agricultural Genomics Institute at Shenzhen, Chinese Academy of Agricultural Sciences, Shenzhen (CN)

(72) Inventors: Kui LI, Shenzhen (CN); Yulian MU, Shenzhen (CN); Lei HUANG, Shenzhen (CN); Yulong ZHU, Shenzhen (CN)

(21) Appl. No.: 18/919,096

(22) Filed: Oct. 17, 2024

(30) Foreign Application Priority Data
Dec. 25, 2023 (CN) 202311796518.6

(51) Int. Cl. C12Q 1/6888; C12Q 1/683; C12Q 1/686

(52) U.S. Cl. CPC C12Q 1/6888; C12Q 1/683; C12Q 1/686; C12Q 2600/156; C12Q 2600/16

(57) ABSTRACT

A reagent, a kit, and a method for detecting a pig economic trait-associated mutation site are provided, belonging to the technical field of molecular markers. The reagent for detecting a pig economic trait-associated mutation site is provided, and the reagent and an endonuclease are prepared into the kit for detecting a pig economic trait-associated mutation site. The method for detecting a pig economic trait-associated mutation site is also provided. After PCR amplification, gene fragments of WIP1, TRIM55, and GAS7 of a pig show site differences between different individuals. Restriction endonucleases are used for digestion, and PCR or multiplex PCR analysis is conducted to identify single nucleotide polymorphisms (SNPs) of the pig, such that the wild type, the homozygous mutant type, and the heterozygous mutant type that are difficult to differentiate in morphology are differentiated from the molecular biology level.

### CROSS REFERENCE TO RELATED APPLICATION

This patent application claims the benefit and priority of Chinese Patent Application No. 202311796518.6 filed with the China National Intellectual Property Administration on Dec. 25, 2023, the disclosure of which is incorporated by reference herein in its entirety as part of the present application.

### REFERENCE TO SEQUENCE LISTING

A computer readable XML file entitled “GWP20240805844_seqlist”, that was created on Sep. 18, 2024, with a file size of about 18,851 bytes, contains the sequence listing for this application, has been filed with this application, and is hereby incorporated by reference in its entirety.

### TECHNICAL FIELD

The present disclosure belongs to the technical field of molecular markers, and particularly relates to a reagent, a kit, and a method for detecting a pig economic trait-associated mutation site.

### BACKGROUND

Single nucleotide polymorphism (SNP) mainly refers to DNA sequence polymorphisms caused by variations in a single nucleotide at the genome level. These variations may affect the DNA transcription and then affect protein synthesis, ultimately changing the metabolic process and affecting individual traits. Therefore, SNP detection is extremely important in livestock production. Currently, the main methods for SNP detection include sequencing, chip-based method, enzymatic detection, high-resolution melting (HRM) method, denaturing high-performance liquid chromatography (DHPLC), and matrix-assisted laser desorption ionization time-of-flight mass spectrometry (MALDI-TOF-MS).

Restriction fragment length polymorphism (RFLP), belonging to the enzymatic detection, is caused by individual gene mutations that lead to changes in the restriction sites, thus ultimately resulting in differences in the number and length of restriction fragments. The RFLP can be adopted to detect mutations in DNA sequences. When the DNA sequences from different individuals are digested with the same enzyme and subjected to gel electrophoresis, variations in the numbers and sizes of fragments can be observed. RFLP is generally used to diagnose diseases in human body, such as sickle cell anemia in human beings. RFLP is also used in animal genetic breeding. Polymerase chain reaction-restriction fragment length polymorphism (PCR-RFLP), as a classic genotype detection method, offers advantages such as simplicity, stability of results, desirable specificity, high sensitivity, and low requirements for sample quality. The PCR-RFLP technology is based on PCR, where the genomic DNA is firstly amplified with two specific primers, followed by digestion of a resulting amplified product with suitable restriction endonucleases, and then the gene polymorphism is detected by electrophoresis.

So far, PCR-RFLP has been widely used in pig breeding practice. As early as 1995, researchers used PCR-RFLP to identify the genotype of a pig halothane gene using a few strands of pig hairs. In 1997, researchers employed PCR-RFLP to identify the genotype of Ryanodine receptor 1 gene (RTR1). From 2006 to 2009, researchers utilized PCR-RFLP to identify the polymorphism of an estrogen receptor gene and a follicle stimulating hormone β subunit gene in Chinese native pig breeds, Landrace pigs, and Large White pigs, and then studied the influence of these two genes on the survival rate of weaned piglets. This suggests that PCR-RFLP plays a significant role in genetic breeding of pigs.

However, the current SNP detection technology based on PCR-RFLP can only detect one SNP in one test, while a certain growth trait of pigs is always related to multiple sites. When multiple sites need to be tested, a sequence of each site needs to be PCR amplified, enzyme digested, and electrophoresed separately. Therefore, there is an urgent need to develop a technology that can achieve simultaneous detection of multiple sites.

### SUMMARY

An objective of the present disclosure is to provide a reagent, a kit, and a method for detecting a pig economic trait-associated mutation site. This method allows for the differentiation of morphologically similar wild type, homozygous mutant, and heterozygous mutant without the need for sequencing, relying solely on molecular biology techniques. It is characterized by its simplicity of operation and intuitive identification advantages.

The present disclosure provides a reagent for detecting a pig economic trait-associated mutation site, including a specific primer pair designed for a mutation site of at least one gene selected from the group consisting of WIP1, TRIM55, and GAS7; where

the WIP1 includes the sequence of SEQ ID NO: 1, the TRIM55 includes the sequence of SEQ ID NO: 2, and the GAS7 includes the sequence of SEQ ID NO: 3.

In some embodiments, a WIP1-specific primer pair includes WIP1-F having the nucleotide sequence of SEQ ID NO: 4 and WIP1-R having the nucleotide sequence of SEQ ID NO: 5;

a TRIM55-specific primer pair includes TRIM55-F having the nucleotide sequence of SEQ ID NO: 6 and TRIM55-R having the nucleotide sequence of SEQ ID NO: 7; and

a GAS7-specific primer pair includes GAS7-F having the nucleotide sequence of SEQ ID NO: 8 and GAS7-R having the nucleotide sequence of SEQ ID NO: 9.

The present disclosure further provides a kit for detecting a pig economic trait-associated mutation site, including the above reagent for detection and a restriction endonuclease corresponding to a restriction site on the specific primer pair.

In some embodiments, the restriction endonuclease is selected from the group consisting of ScaI-HF (NEB, R3122V), BstUI (NEB, R0518V), and HindIII (NEB, R0104V).

The present disclosure further provides a method for detecting a pig economic trait-associated mutation site, including amplifying at least one gene fragment of genes WIP1, TRIM55, and GAS7 of a pig by PCR or multiplex PCR, digesting a resulting amplified product of each gene with a restriction endonuclease, and determining an SNP type of at least one of the genes WIP1, TRIM55, and GAS7 of the pig based on a resulting digested fragment; where

a specific primer pair for the amplifying is derived from the reagent or the kit for detecting a pig economic trait-associated mutation site; and

the restriction endonuclease is derived from the kit for detecting a pig economic trait-associated mutation site.

In some embodiments, a procedure of the PCR includes: initial denaturation at 95° C. for 3 min; 30 to 35 cycles of denaturation at 95° C. for 15 s, annealing at 50° C. for 15 s, and extension at 72° C. for 5 s; and final extension at 72° C. for 3 min.

In some embodiments, a system of the PCR has a volume of 50 μL and includes: 25 μL of 2×Rapid Taq Master Mix (Vazyme, P222), 1 μL of each of upstream and downstream primers at 10 μM, 10 ng of a template, and H₂O as a balance.

In some embodiments, a procedure of the multiplex PCR includes: initial denaturation at 95° C. for 3 min; 30 to 35 cycles of denaturation at 95° C. for 30 s, annealing at 50° C. for 90 s, and extension at 72° C. for 30 s; and final extension at 72° C. for 10 min.

In some embodiments, a system of the multiplex PCR has a volume of 50 μL and includes: 25 μL of 2×Multiplex Buffer (Vazyme, PM101), 1 μL of Multiplex DNA Polymerase (Vazyme, PM101), 0.5 μL of each of primers at 10 μM, 10 ng of a template, and H₂O as a balance.

The present disclosure further provides use of the reagent or the kit for detecting a pig economic trait-associated mutation site in differentiation for an SNP site of a pig economic trait into a wild type, a homozygous mutant type, and a heterozygous mutant type.

Beneficial effects: the present disclosure provides a reagent for detecting a pig economic trait-associated mutation site, and the reagent and an endonuclease are prepared into a kit for detecting the pig economic trait-associated mutation site. Based on previous reports, together with the research of our team, three SNP sites are selected as detection targets, namely: rs_334589942 site (T>G) in the WIP1 (also known as PPM1D)) gene related to pig thickness of backfat; rs_326263523 site (A>G) of the TRIM55 gene related to leg-to-hip ratio and thickness of backfat; and rs_81437379 site (T>C) of the GAS7 gene related to loin-eye thickness. After PCR amplification, gene fragments of genes WIP1, TRIM55, and GAS7 of a pig show site differences between different individuals. Restriction endonucleases are used for digestion, and PCR or multiplex PCR analysis is conducted to identify SNPs of the pig, such that a wild type, a homozygous mutant type, and a heterozygous mutant type that are difficult to differentiate in morphology are differentiated from the molecular biology level. The detection method has the advantages of simple operation, intuitive identification, and no need for sequencing.

### BRIEF DESCRIPTION OF THE DRAWINGS

FIG. 1 shows the correspondence between the genotype of the mutation site and the length of the restriction fragment of different genotypes of each gene;

FIGS. 2A-2C show expected electrophoresis image, electrophoresis image, and sequencing results of samples for digestion of the WIP1 gene (SEQ ID NO: 11 for GG genotype, and SEQ ID NO: 12 for TG genotype);

FIGS. 3A-3C show expected electrophoresis image, electrophoresis image, and sequencing results of samples for digestion of the TRIM55 gene (SEQ ID NO: 13 for AG genotype, and SEQ ID NO: 14 for AA genotype);

FIGS. 4A-4C show expected electrophoresis image, electrophoresis image, and sequencing results of samples for digestion of the GAS7 gene (SEQ ID NO: 15 for TT genotype, and SEQ ID NO: 16 for TC genotype);

FIGS. 5A-5C show expected electrophoresis image, electrophoresis image, and sequencing results of samples for double digestion of the WIP1 (SEQ ID NO: 11 for GG genotype, and SEQ ID NO: 12 for TG genotype) and GAS7 genes (SEQ ID NO: 15 for TT genotype, and SEQ ID NO: 16 for TC genotype);

FIGS. 6A-6C show expected electrophoresis image, electrophoresis image, and sequencing results of samples for double digestion of the WIP1 (SEQ ID NO: 11 for GG genotype, and SEQ ID NO: 12 for TG genotype) and TRIM55 genes (SEQ ID NO: 13 for AG genotype, and SEQ ID NO: 14 for AA genotype);

FIGS. 7A-7C show expected electrophoresis image, electrophoresis image, and sequencing results of samples for double digestion of the GAS7 (SEQ ID NO: 15 for TT genotype, and SEQ ID NO: 16 for TC genotype) and TRIM55 genes (SEQ ID NO: 13 for AG genotype, and SEQ ID NO: 14 for AA genotype); and

FIGS. 8A-8C show expected electrophoresis image, electrophoresis image, and sequencing results of samples for triple digestion of the WIP1 (SEQ ID NO: 11 for GG genotype, and SEQ ID NO: 12 for TG genotype), TRIM55 (SEQ ID NO: 13 for AG genotype, and SEQ ID NO: 14 for AA genotype), and GAS7 (SEQ ID NO: 15 for TT genotype, and SEQ ID NO: 16 for TC genotype) genes.

### DETAILED DESCRIPTION OF THE EMBODIMENTS

The present disclosure provides a reagent for detecting a pig economic trait-associated mutation site, including a specific primer pair designed for a mutation site of at least one gene selected from the group consisting of WIP1, TRIM55, and GAS7; where

The WIP1 includes the sequence of SEQ ID NO: 1 (AGGAATCAACCCTGCTTACATCTGGATTTCAAACTTCTGGCCTCCAAAATCATGAGA GAACAAATTTCCGTTGTTTTAAGCCACCCAATTTCTGCTAATTTGTGACAGCACCTCT ACAAAACAAATACAGCAATTCTGTTCCTCAAATTTTTTACATAACTAGTTTTGTAACAT TTATGATGTTTCTTGGCTGAACAATTGATTATGATGGTTATCAAATGTGAATTTTTAGTT CCATCATTCCTTCTACATTCATCAGCGGACTTTTATAAAGTAATGATGATTAACAAAAA TGGGGAAAAACAAGT^{Λ}ACKTGTCAAGTAGGGCAAATTCGTTCAGAACATATGATGTG TGAGAGGGTGAAGG; where the bold part represents the SNP site and its mutation type, the underlined part represents the ScaI-HF endonuclease recognition sequence, the recognition sequence is AGT^{Λ}ACT, ^{Λ} represents the restriction site), and K represents T/G. The TRIM55 includes the sequence of SEQ ID NO: 2 (ACAAGAGTGTAGTGCCGACTTTTTTCAACACTTGCCACTTTTACTGACR^{Λ}CGCACAC ACACACATACCACACCTACACACCCTTTGTAATGTTTCCCACACGATAAAGATTTGCAT AAGATCAAGTTCCCCTTTTTTTCCTAATGCCTTCTGCAACTATTTTCTCAAAGCATGGT TTTCCCTCACTGTGTTAGATAAGTCATCTGGAATTAAAAGGTATCTTTTATAGAAATCC TGTTCAGAGGTCCC; where the bold part represents the SNP site and its mutation type, the underlined part represents the BstUI endonuclease recognition sequence, the recognition sequence is CG^{Λ}CG, ^{Λ} represents the restriction site), and R represents A/G. The GAS7 includes the sequence of SEQ ID NO: 3 (GAAGCCAGTTAACAGGA^{Λ}AACYTGCCACTCCCTGGGAGACGTCAGCCACTTTCCAA CCACCTCCCTCCAGCCTTCCCCCTTGGGGTCCAGCCCTCCAGAAACAACAGACCGTC CTCCTCCCCCCTCCCCCACAAGCCAGGAGAAGGCCAAGGGCACGGGACTCTGCGAG GATGGATCACGCCAGCGGCAGGTCTGCGCCGGATCCCTCTCCACCCTGCCCGCCTCT CACGCCGGCCCTCATTTGCAGGAGACGTGCCGCTGTAGCTGAGTGTATATATCGTCTG GATGATTTGCCGTGG; where the bold part represents the SNP site and its mutation type, the underlined part represents the HindIII endonuclease recognition sequence, the recognition sequence is A^{Λ}AGCTT, ^{Λ} represents the restriction site), and Y represents T/C. Since there is no suitable restriction site near the SNP site of the GAS7 gene, a base is artificially changed in the upstream primer during primer design to form a suitable restriction site for the SNP. That is, the original primer sequence GAAGCCAGTTAACAGGAAAC (SEQ ID NO: 10) is changed to GAAGCCAGTTAACAGGAAGC (SEQ ID NO: 8), and the restriction site exists after the penultimate base is changed from A to G (5′-A^{Λ}AGCTT-3′).

In the present disclosure, the following primer pairs are designed and synthesized by a biological company in accordance with the above sequences:

WIP1-F (SEQ ID NO: 4):
5′ AGGAATCAACCCTGCTTA 3′;
WIP1-R (SEQ ID NO: 5):
5′ CCTTCACCCTCTCACACA 3′;
TRIM55-F (SEQ ID NO: 6):
5′ GGGACCTCTGAACAGGATTT 3′;
TRIM55-R (SEQ ID NO: 7):
5′ ACAAGAGTGTAGTGCCGACTT 3′;
GAS7-F (SEQ ID NO: 8):
5′ GAAGCCAGTTAACAGGAAGC 3′;
GAS7-R (SEQ ID NO: 9):
5′ CCACGGCAAATCATCCA 3′.

The present disclosure further provides a reagent for detecting a pig economic trait-associated mutation site, including the above reagent and a restriction endonuclease corresponding to a restriction site on the specific primer pair.

In the present disclosure, the restriction endonuclease is preferably selected from the group consisting of ScaI-HF, BstUI, and HindIII. In the examples, ScaI-HF is combined with WIP1, BstUI is combined with TRIM55, and HindIII is combined with GAS7 for the detection of pig economic trait-associated mutation sites.

The present disclosure further provides a method for detecting a pig economic trait-associated mutation site, including amplifying at least one gene fragment of genes WIP1, TRIM55, and GAS7 of a pig by PCR or multiplex PCR, digesting a resulting amplified product of each gene with a restriction endonuclease, and determining an SNP type of at least one of the genes WIP1, TRIM55, and GAS7 of the pig based on a resulting digested fragment; where

a specific primer pair for the amplifying is derived from the detection reagent or the reagent for detecting a pig economic trait-associated mutation site; and

the restriction endonuclease is derived from the reagent for detecting a pig economic trait-associated mutation site.

In the present disclosure, the primers can be used for both conventional PCR amplification and multiplex PCR amplification. For conventional PCR amplification, the amplification is conducted in a 50 μL system including: 25 μL of 2×Rapid Taq Master Mix, 1 μL of each of upstream and downstream primers at 10 μM, 10 ng of a template, and H₂O as a balance; procedures of the PCR include: initial denaturation at 95° C. for 3 min; 30 to 35 cycles of denaturation at 95° C. for 15 s, annealing at 50° C. for 15 s, and extension at 72° C. for 5 s; and final extension at 72° C. for 3 min. For multiplex PCR amplification, the amplification is conducted in a 50 μL system including: 25 μL of 2×Multiplex Buffer, 1 μL of Multiplex DNA Polymerase, 0.5 μL of each of primers at 10 μM, 10 ng of a template, and H₂O as a balance; procedures of the multiplex PCR include: initial denaturation at 95° C. for 3 min; 30 to 35 cycles of denaturation at 95° C. for 30 s, annealing at 50° C. for 90 s, and extension at 72° C. for 30 s; and final extension at 72° C. for 10 min.

In an embodiment of the present disclosure, tissue DNA is preferably used as a template to extract the cell genome using Fast Pure Cell/Tissue DNA Isolation Mini Kit (Vazyme, DC102), which specifically includes the followings:

### (1) Tissue Digestion and Lysis

i) One microgram to 20 mg of chopped or ground tissue sample is placed into a 1.5 mL centrifuge tube, 230 μL of Buffer GA and 20 μL of PK working solution are added into the sample, mixed well by vortex, and placed in a 55° C. water bath until complete enzymatic hydrolysis (tissue samples require 0.5 h to 3 h).

ii) Twenty hundred and fifty microliters of Buffer GB is added into a resulting cell suspension and mixed well by vortex. A resulting mixture is incubated in a 70° C. water bath for 10 min.

### (2) Column Purification

iii) One hundred and eighty microliters of anhydrous ethanol is added into a resulting digestion solution and mixed well by vortex for 15 s to 20 s.

iv) A gDNA purification column is placed into a 2 mL collection tube. A mixed solution (supernatant) obtained in the previous step is transferred to the purification column. Centrifugation is conducted at 13,400 rpm for 1 min.

v) The filtrate is discarded and the column is placed in a collection tube. 500 μL of Washing Buffer A is added into the purification column. Centrifugation is conducted at 13,400 rpm for 1 min.

vi) The filtrate is discarded and the purification column is placed in a collection tube. Six hundred and fifty microliters of Washing Buffer B is added into the purification column. Centrifugation is conducted at 13,400 rpm for 1 min.

vii) Step iv) is repeated.

viii) The filtrate is discarded and the purification column is placed in a collection tube. Centrifugation is conducted at 13,400 rpm for 2 min on the empty tube.

ix) The purification column is placed in a new 1.5 mL centrifuge tube. One hundred microliters of Elution Buffer preheated to 70° C. is added into the center of the membrane in the purification column and allowed to stand at room temperature for 3 min. Centrifugation is conducted at 13,400 rpm for 1 min.

x) The purification column is removed, and a resulting DNA is stored at 2° C. to 8° C., optionally placed at −20° C. to allow long-term storage.

In the present disclosure, the extracted genomic DNA is used as a template for PCR amplification according to the above method, and a resulting product is purified and recovered using DNA Clean & Concentrator™-5 (ZYMO RESEARCH, D4014), which specifically includes the followings:

i) Five times volume of DNA binding buffer is added and mixed well by vortex;

ii) a resulting mixture is transferred to a Zymo-Spin Column and centrifuged at 10,000 rpm and room temperature for 30 s;

iii) a resulting product is washed 2 times with 200 μL of DNA wash buffer and centrifuged at 10,000 rpm and room temperature for 30 s; and

iv) Twenty five microliters of DNA Elution buffer is added into the column, incubated at room temperature for 1 min, and centrifuged at 10,000 rpm and room temperature for 30 s.

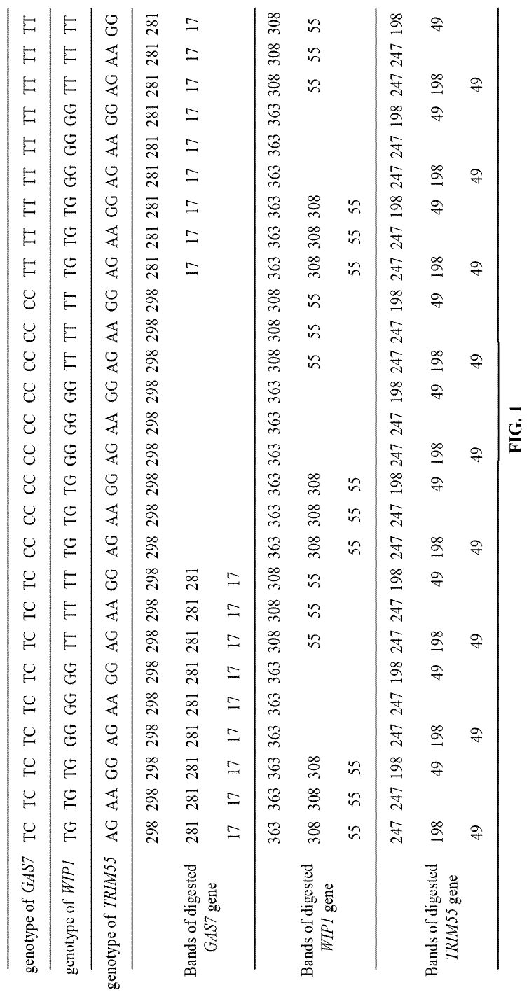

In the present disclosure, the product after purification and recovery is subjected to digestion, which preferably includes: taking 200 ng of the purified PCR product to allow digestion with NEB enzyme. The digestion is performed in a 50 μL system preferably including: 2 U of enzyme (NEB), 5 μL of NEB Buffer (NEB), 200 ng of DNA, and ddH₂O as a balance. After the system is mixed, the reaction is conducted at 37° C. for 0.5 h, and the enzyme is inactivated at 80° C. for 20 min. Ten microliters of 6×DNA loading buffer (Thermo Fisher, R0611) is added into per 50 μL of the system, and 6 μL of the system is taken for gel electrophoresis. The different SNP types of the three genes and their digested fragment lengths are shown in FIG. 1.

In the present disclosure, the gel electrophoresis preferably adopts 8% lyacrylamide gel electrophoresis (PAGE) gel: 1 mm plate, and the gel preparation and additives are shown in Table 1. The electrophoresis is conducted using 0.5×Tris-Borate-EDTA (TBE) electrophoresis buffer, with parameters being: constant voltage: 60 V, 15 min; 120 V, 90 min. After the electrophoresis, Gel staining is conducted, where SYBR Gold (S11494, Invitrogen) is diluted 10,000 times with 1×TBE and the staining is conducted for 5 min. The gel is then subjected to photographing.

TABLE 1
8% PAGE gel

| Material | Volume | | Remarks |
|---|---|---|---|
| 5 × TBE | 2.52 | mL | Solidification time ≥40 min |
| 30% gel making solution | 3.7 | mL | Water-like exudate is observed |
| (Salarbio, A1010) | | | when the remaining gel solidifies |
| 10% APS | 176.4 | μL | |
| TEMED | 8.82 | μL | Finally added in a fume hood |
| ddH₂O | 7.44 | mL | |

The present disclosure further provides use of the reagent or the kit for detecting a pig economic trait-associated mutation site in differentiation for an SNP site of a pig economic trait into a wild type, a homozygous mutant type, and a heterozygous mutant type.

In an embodiment of the present disclosure, the SNP type corresponding to each gene can be determined according to the size of the digested band shown in FIG. 1.

To further illustrate the present disclosure, the reagent, the kit, and the method for detecting a pig economic trait-associated mutation site provided by the present disclosure are described in detail below in connection with examples, but these examples should not be construed as limiting the claimed scope of the present disclosure.

In the examples of the present disclosure, the reagents and biological materials used are all commercial products unless otherwise specified. If no specific conditions are specified in the examples, the examples will be implemented under conventional conditions or the conditions recommended by the manufacturer. The molecular biology experimental methods not specifically described in the examples are all performed with reference to the specific methods listed in the book Molecular Cloning Experiment Guide (fourth edition) by J. Sambrook, or according to the kits and product instructions.

### Example 1

Detection of SNP on rs_334589942 Site of WIP1 Gene

S1, tissue DNA extraction: genomic DNA was extracted from cells using the Fast Pure Cell/Tissue DNA Isolation Mini Kit (Vazyme, DC102).

S2, primer design and DNA amplification:

WIP1-F (SEQ ID NO: 4):
5′ AGGAATCAACCCTGCTTA 3′;
WIP1-R (SEQ ID NO: 5):
5′ CCTTCACCCTCTCACACA 3′.

A 50 μL system included: 25 μL of 2×Rapid Taq Master Mix, 1 μL of upstream primer (10 μM), 1 μL of downstream primer (10 μM), 10 ng of DNA, and ddH₂O as a balance; the procedure included: initial denaturation at 95° C. for 3 min; 30 cycles of denaturation at 95° C. for 15 s, annealing at 50° C. for 15 s, and extension at 72° C. for 5 s; and final extension at 72° C. for 3 min.

S3, product recovery and purification:

The product was purified and recovered using DNA Clean & Concentrator™-5 (ZYMO RESEARCH, D4014).

S4, ScaI-HF digestion:

Two hundred nanograms of the purified PCR product from step S3 was digested with endonuclease ScaI-HF, mixed well, reacted at 37° C. for 30 min, the enzyme was inactivated at 80° C. for 20 min, 10 μL of 6×DNA loading buffer was added into per 50 μL of the system, and 6 μL of the system was taken to allow gel electrophoresis.

S5, Electrophoresis:

i) Gel preparation: 8% PAGE gel: 1 mm plate, gel, other additives are shown in Table 6.

ii) Electrophoresis was conducted, with 0.5×TBE electrophoresis buffer, parameters being: constant voltage: 60 V, 15 min; 120 V, 90 min.

iii) Gel staining was conducted, where SYBR Gold was diluted 10,000 times with 1×TBE and the staining was conducted for 5 min.

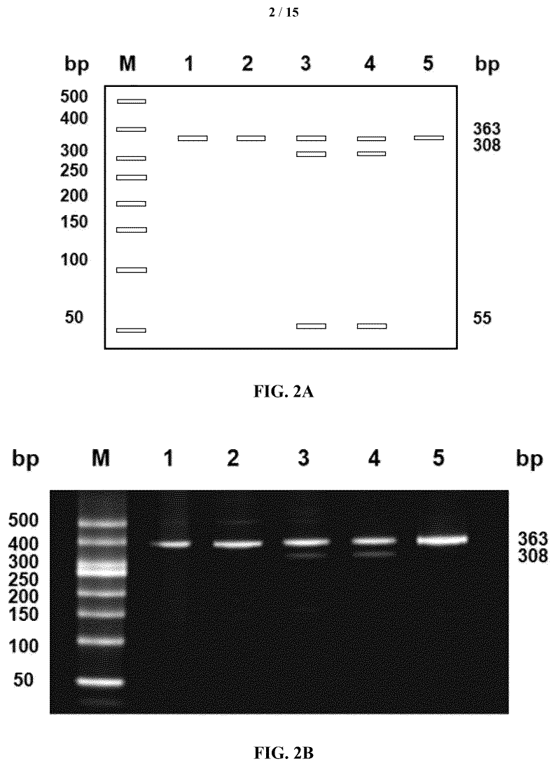

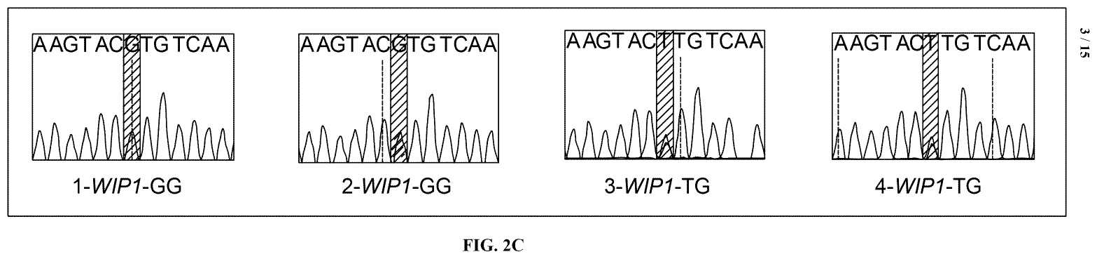

iv) The gel was subjected to photographing. Notes for the photographing process: due to the small molecular weight of one of the digestion products, it is generally not directly observable during photographing after PAGE. However, its existence can be inferred from the larger digested bands. In this experiment, the WIP1 gene PCR product was 363 bp long and could be digested into two bands of 308 bp and 55 bp by enzyme, of which the 55 bp band could not be observed; but when the 308 bp band could be observed, it meant that the product had been digested, and the 55 bp band existed. The GAS7 gene PCR product was 298 bp long and could be digested into two bands of 281 bp and 17 bp by enzyme, of which the 17 bp band could not be observed; but when the 281 bp band could be observed, it meant that the product had been digested, and the 17 bp band existed. The TRIM55 gene PCR product was 247 bp long and could be cut into two bands of 198 bp and 49 bp by enzyme digestion, of which the 49 bp band could not be observed; but if the 198 bp band could be observed, it meant that the product had been digested, and the 49 bp band existed. This method applied to all subsequent experiments and would not be repeated here.) FIG. 2A is an expected electrophoresis image plotted according to the sequencing results, while the electrophoresis image of the actual enzyme digestion result is shown in FIG. 2B, indicating that the bands observable in the actual image corresponded one to one with the prediction image. There was 1 band at 363 bp in lanes 1 and 2, indicating that the rs_334589942 site of the WIP1 gene in samples 1 and 2 was of GG type; 2 bands at 363 bp and 308 bp in lanes 3 and 4 were visible, indicating that the site was of GT type; lane 5 served as a control, showing the undigested PCR product of the WIP1 gene at 363 bp. FIG. 2C is the sequencing result of the PCR product of the WIP1 gene from the sample used in this example, where the shaded portion represented the base type of the rs_334589942 site of the WIP1 gene.

### Example 2

Detection of SNP at rs_326263523 Site of TRIM55 Gene

Except that the primers were the primers of SEQ ID NO: 6 and SEQ ID NO: 7 and BstUI was used for digestion, the rest of the scheme was the same as that in Example 1.

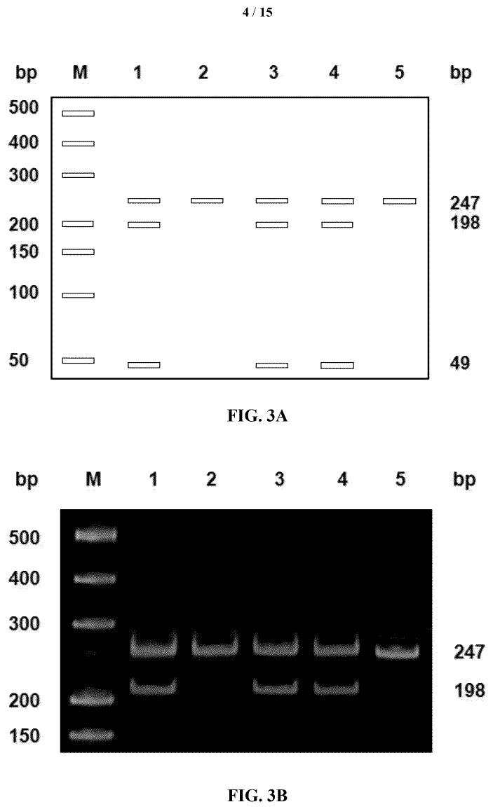

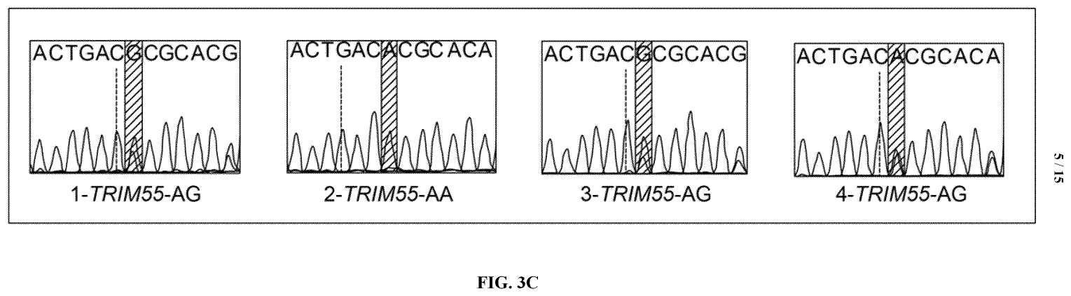

FIG. 3A is an expected electrophoresis image made according to the sequencing results, while the electrophoresis image of the actual enzyme digestion is shown in FIG. 3B, indicating that the bands observable in the actual image corresponded one to one with those in the expected image. The bands in lanes 1, 3, and 4 were identical, with sizes of 247 bp and 198 bp, indicating that the rs_326263523 site of the TRIM55 gene in samples 1, 3, and 4 was of AG type; lane 2 had only one band, with a size of 247 bp, indicating that the site in sample 2 was of AA type; lane 5 was the control group, which was a PCR product of the TRIM55 gene without enzyme digestion, with a band of 247 bp. FIG. 3C shows the sequencing result of the PCR product of the sample TRIM55 gene used in this example, where the shaded portion represented the base type of the rs_326263523 site of the TRIM55 gene.

### Example 3

Detection of SNP in rs_81437379 Site of GAS7 Gene

Except that the primers were the primers of SEQ ID NO: 8 and SEQ ID NO: 9 and HindIII was used for digestion, the rest of the scheme was the same as that in Example 1.

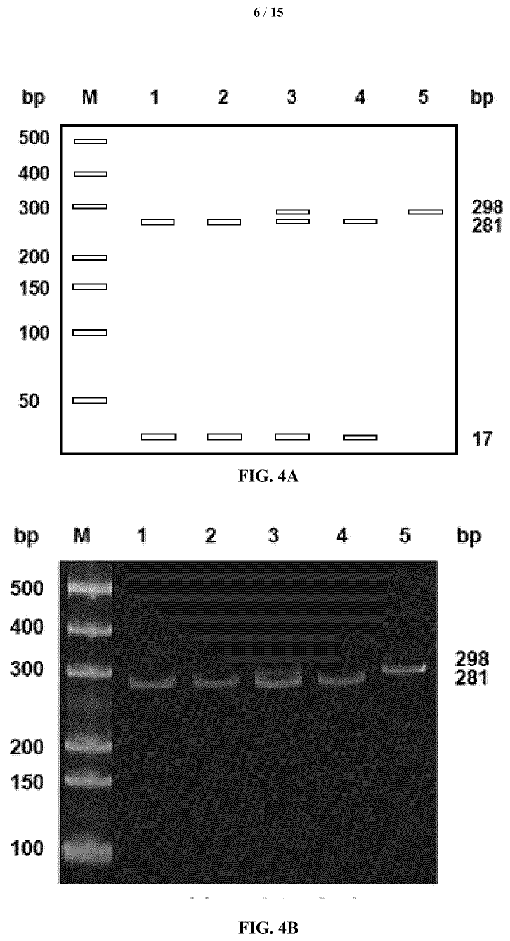

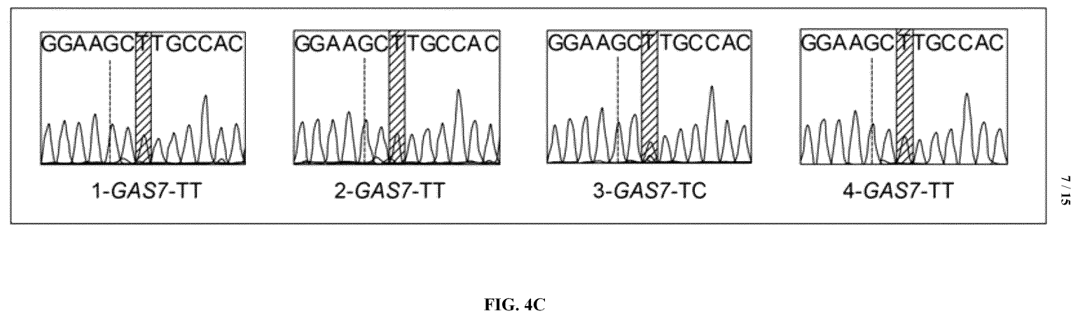

FIG. 4A is an expected electrophoresis image made according to the sequencing results, while the electrophoresis image of the actual enzyme digestion is shown in FIG. 4B, indicating that the bands observable in the actual image corresponded one to one with the expected image. Lanes 1, 2, and 4 each had only one band, 281 bp in size, indicating that the rs_81437379 site of the GAS7 gene in samples 1, 2, and 4 was of TT type; lane 3 had two bands of 298 bp and 281 bp, indicating that the site in sample 3 was of CT type; lane 5 was the control group, which was a PCR product of the GAS7 gene without enzyme digestion, with a band of 298 bp. FIG. 4C shows the sequencing result of the PCR product of the sample GAS7 gene used in this example, where the shaded portion represented the base type of the rs_81437379 site of the GAS7 gene.

### Example 4

Detection SNPs in Both rs_334589942 Site of WIP1 Gene and rs_81437379 Site of GAS7 Gene

Except for the primers of SEQ ID NO: 4 and SEQ ID NO: 5 and the primers of SEQ ID NO: 8 and SEQ ID NO: 9 for duplex PCR amplification, and the HindIII and ScaI-HF were used for double digestion, the rest of the scheme was the same as that in Example 1.

A 50 μL system for multiplex PCR included: 25 μL of 2×Multiplex Buffer, 1 μL of Multiplex DNA Polymerase, 0.5 μL of each of primers (10 μM), 10 ng of DNA, and ddH₂O added to 50 μL. The procedure included: initial denaturation at 95° C. for 3 min; 30 cycles of denaturation at 95° C. for 30 s, annealing at 50° C. for 90 s, and extension at 72° C. for 30 s; and final extension at 72° C. for 10 min.

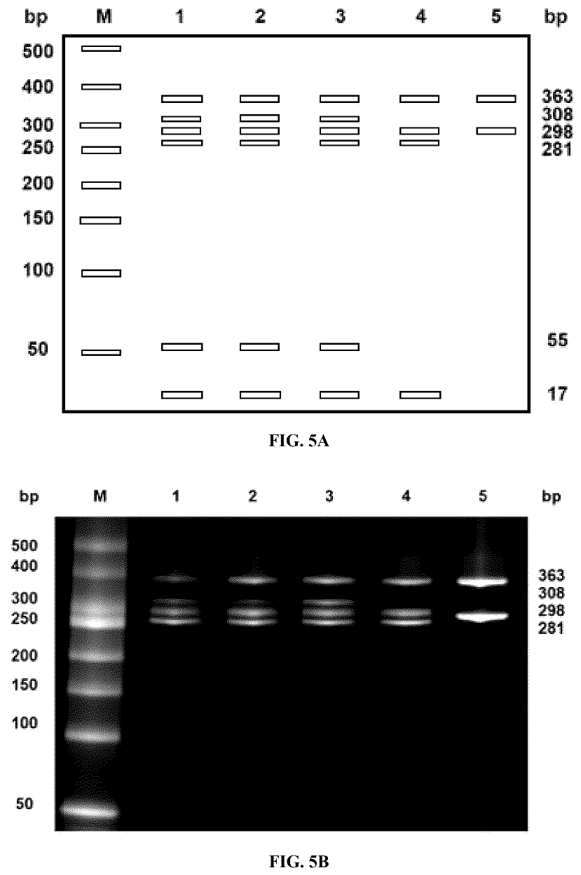

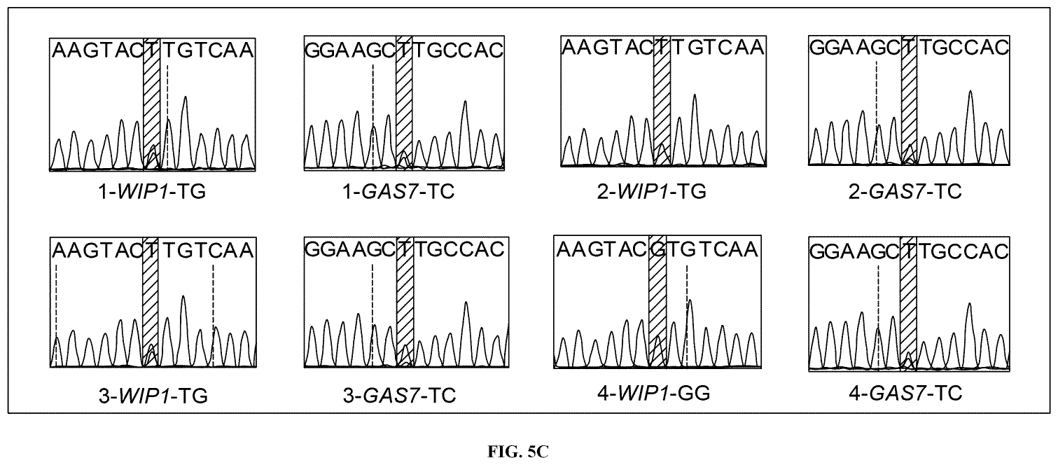

FIG. 5A is an expected electrophoresis image made according to the sequencing results, while the electrophoresis image of the actual enzyme digestion result is shown in FIG. 5B, indicating that the bands observable in the actual image corresponded one to one with the expected image. The bands in lanes 1, 2, and 3 were the same, with sizes of 363 bp, 308 bp, 298 bp, and 281 bp, respectively, indicating that the WIP1 gene rs_334589942 site of samples 1, 2, and 3 was of GT type while the GAS7 gene rs_81437379 site was of CT type; the bands in lane 4 were 363 bp, 298 bp, and 281 bp, indicating that the WIP1 gene rs_334589942 site of sample 4 was of GG type while the GAS7 gene rs_81437379 site was of CT type; lane 5 was the control group, which was a double PCR product of WIP1 and GAS7 genes without enzyme digestion, with bands of 363 bp and 298 bp, respectively. FIG. 5C shows the sequencing result of the PCR product of the sample WIP1 and GAS7 genes used in this example, where the shaded portion represented the base type of the WIP1 gene rs_334589942 site or the GAS7 gene rs_81437379 site.

### Example 5

Detection of SNPs in Both rs_334589942 Site of WIP1 Gene and rs_326263523 Site of TRIM55 Gene

Except for the primers shown in SEQ ID NO: 4 and SEQ ID NO: 5 and the primers shown in SEQ ID NO: 6 and SEQ ID NO: 7 for duplex PCR amplification, and the ScaI-HF and BstUI were used for double digestion, the rest of the scheme was the same as that in Example 4.

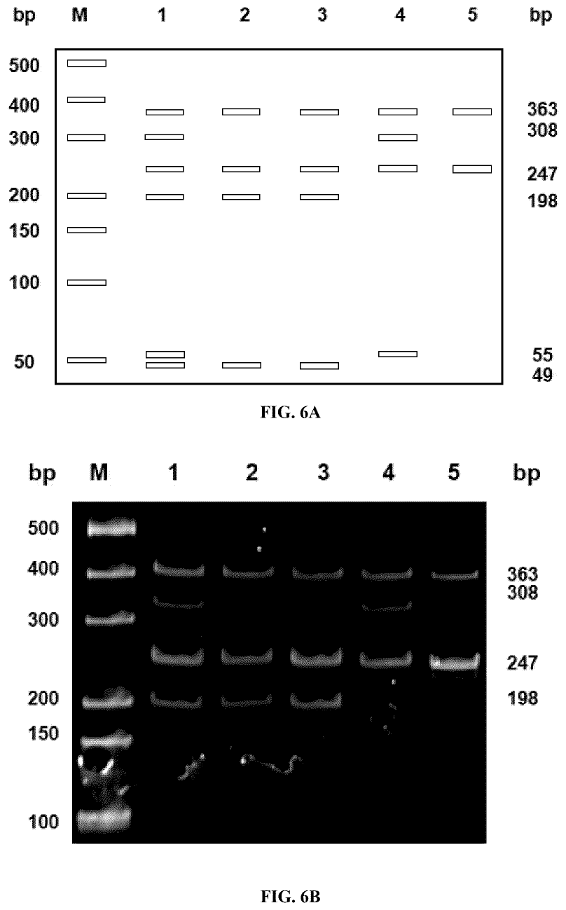

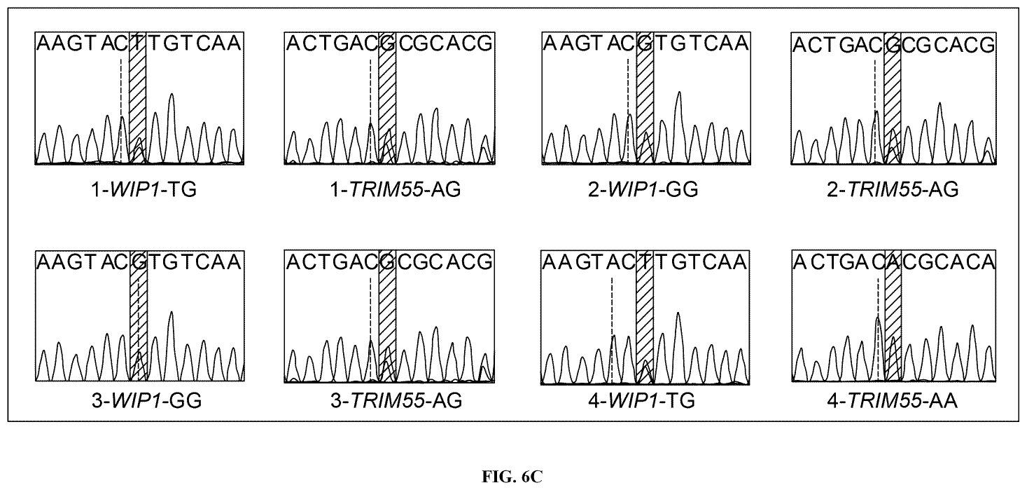

FIG. 6A was an expected electrophoresis image made according to the sequencing results, while the electrophoresis image of the actual enzyme digestion result was shown in FIG. 6B, indicating that the bands observable in the actual image corresponded one to one with the prediction image. The bands in lane 1 were 363 bp, 308 bp, 247 bp, and 198 bp, indicating that the WIP1 gene rs_334589942 site of sample 1 was of GT type while the TRIM55 gene rs_326263523 site was of AG type; the bands in lanes 2 and 3 were identical, with sizes of 363 bp, 247 bp, and 198 bp, indicating that the WIP1 gene rs_334589942 site of samples 2 and 3 was of GG type while the TRIM55 gene rs_326263523 site was of AG type; the bands in lane 4 were 363 bp, 308 bp, and 247 bp, indicating that the WIP1 gene rs_334589942 site of sample 4 was of GT type while the TRIM55 gene rs_326263523 site was of AA type; lane 5 was the control group, which was a double PCR product of WIP1 and TRIM55 genes without enzyme digestion, with bands of 363 bp and 247 bp, respectively. FIG. 6C shows the sequencing result of the PCR product of the sample WIP1 and GAS7 genes used in this example, where a shaded portion represented the base type of the WIP1 gene rs_334589942 site or the GAS7 gene rs_81437379 site.

### Example 6

Detection of SNPs in Both rs_81437379 Site of GAS7 Gene and rs_326263523 Site of TRIM55 Gene

Except for the primers of SEQ ID NO: 6 and SEQ ID NO: 7 and the primers of SEQ ID NO: 8 and SEQ ID NO: 9 for duplex PCR amplification, and the HindIII and BstUI were used for double digestion, the rest of the scheme was the same as that in Example 4.

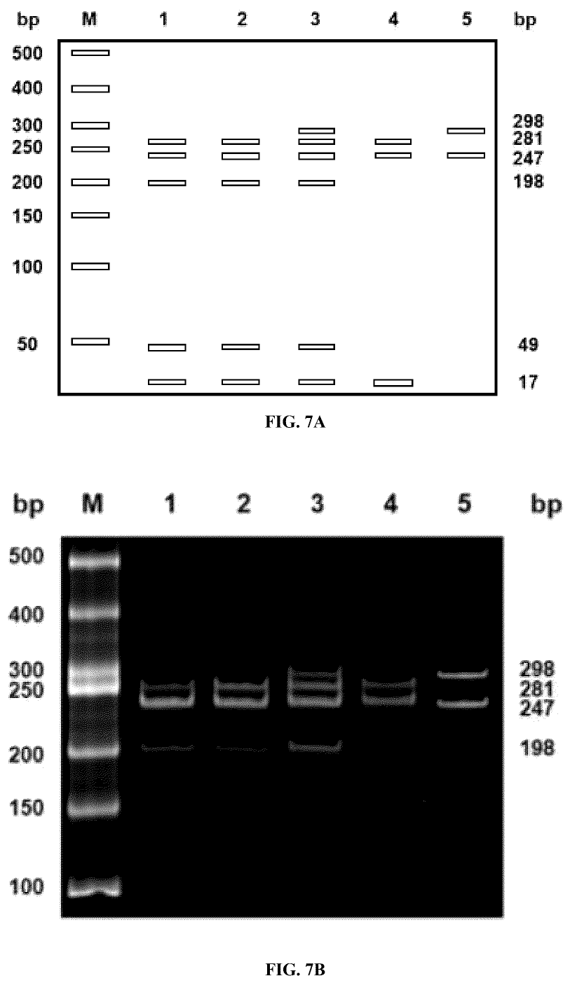

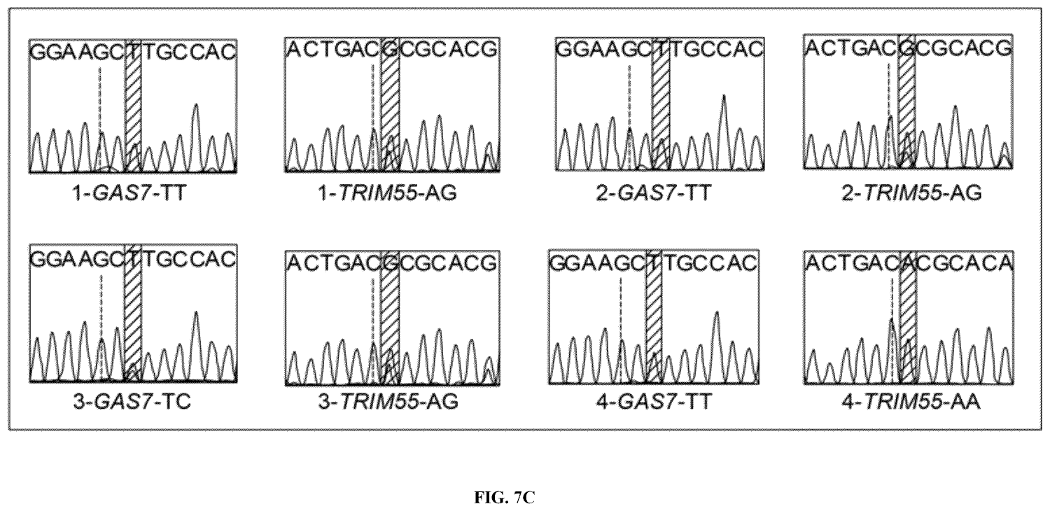

FIG. 7A is an expected electrophoresis image made according to the sequencing results, while the electrophoresis image of the actual enzyme digestion is shown in FIG. 7B, indicating that the bands observable in the actual image corresponded one to one with the expected image. The bands in lanes 1 and 2 were the same, with sizes of 281 bp, 247 bp, and 198 bp, indicating that the GAS7 gene rs_81437379 site of samples 1 and 2 was of TT type while the TRIM55 gene rs_326263523 site was of AG type; the bands in lane 3 were 298 bp, 281 bp, 247 bp, and 198 bp, indicating that the GAS7 gene rs_81437379 site of sample 3 was of TC type while the TRIM55 gene rs_326263523 site was of AG type; the bands in lane 4 were 281 bp and 247 bp, indicating that the GAS7 gene rs_81437379 site of sample 4 was of TT type while the TRIM55 gene rs_326263523 site was of AA type; lane 5 was the control group, which was a double PCR product of GAS7 and TRIM55 genes without enzyme digestion, with bands of 298 bp and 247 bp, respectively. FIG. 7C shows the sequencing result of the PCR product of the sample WIP1 and GAS7 genes used in this example, where the shaded portion represented the base type of the GAS7 gene rs_81437379 site or the TRIM55 gene rs_326263523 site.

### Example 7

Detection of SNPs in Three Sites, rs_334589942 of WIP1 Gene, rs_81437379 of GAS7 Gene, and rs_326263523 of TRIM55 Gene

Except for the primers of SEQ ID NO: 4 and SEQ ID NO: 5, the primers of SEQ ID NO: 6 and SEQ ID NO: 7, and the primers of SEQ ID NO: 8 and SEQ ID NO: 9 for triplex PCR amplification, and the ScaI-HF, HindIII, and BstUI were used for triple digestion, the rest of the scheme was the same as that in Example 4.

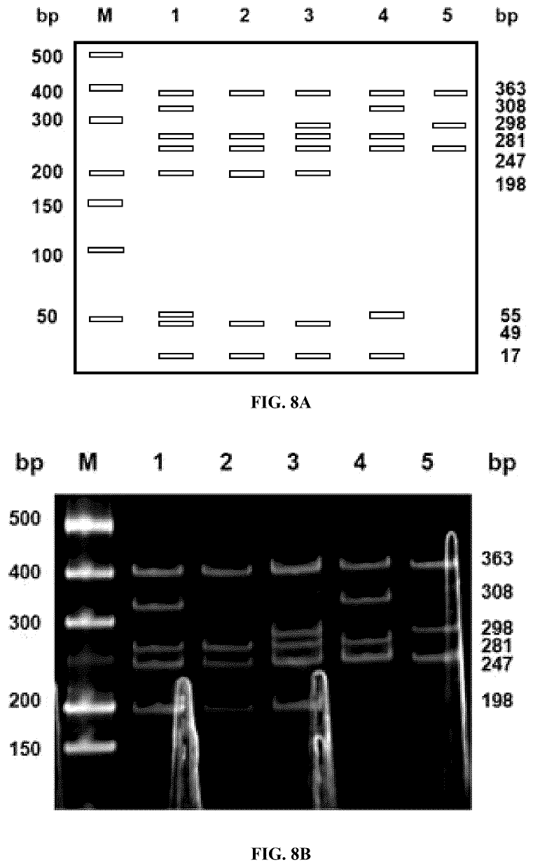

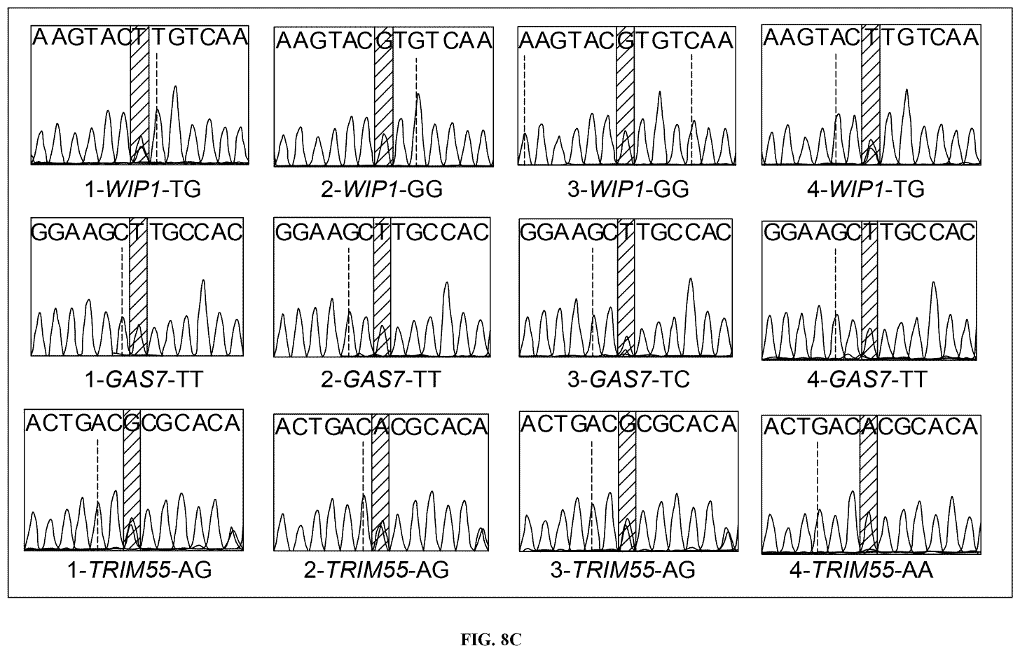

FIG. 8A is an expected electrophoresis image made according to the sequencing results, while the electrophoresis image of the actual enzyme digestion is shown in FIG. 8B, indicating that the bands observable in the actual image corresponded one to one with the expected image. The bands in lane 1 were 363 bp, 308 bp, 281 bp, 247 bp, and 198 bp, indicating that the WIP1 gene rs_334589942 site of sample 1 was of TG type, the GAS7 gene rs_81437379 site was of TT type, and the TRIM55 gene rs_326263523 site was of AG type; the bands in lane 2 were 363 bp, 281 bp, 247 bp, and 198 bp, indicating that the WIP1 gene rs_334589942 site of sample 2 was of GG type, the GAS7 gene rs_81437379 site was of TT type, and the TRIM55 gene rs_326263523 site was of AG type; the bands in lane 3 were 363 bp, 298 bp, 281 bp, 247 bp, and 198 bp, indicating that the WIP1 gene rs_334589942 site of sample 3 was of GG type, the GAS7 gene rs_81437379 site was of TC type, and the TRIM55 gene rs_326263523 site was of AG type; the bands in lane 4 were 363 bp, 308 bp, 281 bp, and 247 bp, indicating that the WIP1 gene rs_334589942 site of sample 4 was of TG type, the GAS7 gene rs_81437379 site was of TT type, and the TRIM55 gene rs_326263523 site was of AA type; lane 5 was the control group, which was a triple PCR product of WIP1, GAS7, and TRIM55 genes without enzyme digestion, with bands of 363 bp, 298 bp, and 247 bp, respectively. FIG. 8C shows the sequencing result of the PCR product of the sample WIP1, GAS7, and TRIM55 genes used in this example, where the shaded portion represented the base type of the WIP1 gene rs_334589942 site, or GAS7 gene rs_81437379 site, or TRIM55 gene rs_326263523 site.

Although the above example has described the present disclosure in detail, it is only a part of, not all of, the embodiments of the present disclosure. Other embodiments may also be obtained by persons based on the example without creative efforts, and all of these embodiments shall fall within the protection scope of the present disclosure.

What is claimed is:

1. A reagent for detecting a pig economic trait-associated mutation site, comprising a specific primer pair designed for a mutation site of at least one gene selected from the group consisting of WIP1, TRIM55, and GAS7; wherein
the WIP1 comprises the sequence of SEQ ID NO: 1, the TRIM55 comprises the sequence of SEQ ID NO: 2, and the GAS7 comprises the sequence of SEQ ID NO: 3.

2. The reagent according to claim 1, wherein a WIP1-specific primer pair comprises WIP1-F having the nucleotide sequence of SEQ ID NO: 4 and WIP1-R having the nucleotide sequence of SEQ ID NO: 5;
a TRIM55-specific primer pair comprises TRIM55-F having the nucleotide sequence of SEQ ID NO: 6 and TRIM55-R having the nucleotide sequence of SEQ ID NO: 7; and
a GAS7-specific primer pair comprises GAS7-F having the nucleotide sequence of SEQ ID NO: 8 and GAS7-R having the nucleotide sequence of SEQ ID NO: 9.

3. A kit for detecting a pig economic trait-associated mutation site, comprising the reagent according to claim 1 and a restriction endonuclease corresponding to a restriction site on the specific primer pair.

4. The kit according to claim 3, wherein a WIP1-specific primer pair comprises WIP1-F having the nucleotide sequence of SEQ ID NO: 4 and WIP1-R having the nucleotide sequence of SEQ ID NO: 5;
a TRIM55-specific primer pair comprises TRIM55-F having the nucleotide sequence of SEQ ID NO: 6 and TRIM55-R having the nucleotide sequence of SEQ ID NO: 7; and
a GAS7-specific primer pair comprises GAS7-F having the nucleotide sequence of SEQ ID NO: 8 and GAS7-R having the nucleotide sequence of SEQ ID NO: 9.

5. The kit according to claim 3, wherein the restriction endonuclease is selected from the group consisting of ScaI-HF, BstUI, and HindIII.

6. A method for detecting a pig economic trait-associated mutation site, comprising amplifying at least one gene fragment of genes WIP1, TRIM55, and GAS7 of a pig by PCR or multiplex PCR, digesting a resulting amplified product of each gene with a restriction endonuclease, and determining a genotype of at least one of the genes WIP1, TRIM55, and GAS7 of the pig based on a resulting digested fragment; wherein
a specific primer pair for amplification is derived from the reagent according to claim 1; and
the restriction endonuclease is derived from a kit.

7. The method according to claim 6, wherein a WIP1-specific primer pair comprises WIP1-F having the nucleotide sequence of SEQ ID NO: 4 and WIP1-R having the nucleotide sequence of SEQ ID NO: 5;
a TRIM55-specific primer pair comprises TRIM55-F having the nucleotide sequence of SEQ ID NO: 6 and TRIM55-R having the nucleotide sequence of SEQ ID NO: 7; and
a GAS7-specific primer pair comprises GAS7-F having the nucleotide sequence of SEQ ID NO: 8 and GAS7-R having the nucleotide sequence of SEQ ID NO: 9.

8. The method according to claim 6, wherein the kit comprises the reagent and a restriction endonuclease corresponding to a restriction site on the specific primer pair.

9. The method according to claim 8, wherein the restriction endonuclease corresponding to a restriction site on the specific primer pair is selected from the group consisting of ScaI-HF, BstUI, and HindIII.

10. The method according to claim 6, wherein procedures of the PCR comprise: initial denaturation at 95° C. for 3 min; 30 to 35 cycles of denaturation at 95° C. for 15 s, annealing at 50° C. for 15 s, and extension at 72° C. for 5 s; and final extension at 72° C. for 3 min.

11. The method according to claim 6, wherein the PCRis performed in a 50 μL system comprising 25 μL of 2×Rapid Taq Master Mix (Vazyme, P222), 1 μL of each of upstream and downstream primers at 10 μM, 10 ng of a template, and H₂0 as a balance.

12. The method according to claim 11, wherein procedures of the PCR comprise: initial denaturation at 95° C. for 3 min; 30 to 35 cycles of denaturation at 95° C. for 15 s, annealing at 50° C. for 15 s, and extension at 72° C. for 5 s; and final extension at 72° C. for 3 min.

13. The method according to claim 6, wherein procedures of the multiplex PCR comprise:
initial denaturation at 95° C. for 3 min; 30 to 35 cycles of denaturation at 95° C. for 30 s, annealing at 50° C. for 90 s, and extension at 72° C. for 30 s; and final extension at 72° C. for 10 min.

14. The method according to claim 6, wherein the multiplex PCR is performed in a 50 μL system comprising 25 μL of 2×Multiplex Buffer (Vazyme, PM101), 1 μL of Multiplex DNA Polymerase (Vazyme, PM101), 0.5 μL of each of primers at 10 μM, 10 ng of a template, and H₂O as a balance.

15. The method according to claim 14, wherein procedures of the multiplex PCR comprise: initial denaturation at 95° C. for 3 min; 30 to 35 cycles of denaturation at 95° C. for 30 s, annealing at 50° C. for 90 s, and extension at 72° C. for 30 s; and final extension at 72° C. for 10 min.

16. A method for genotyping a pig economic trait gene, comprising applying the reagent according to claim 1.

17. The method according to claim 16, wherein a WIP1-specific primer pair comprises WIP1-F having the nucleotide sequence of SEQ ID NO: 4 and WIP1-R having the nucleotide sequence of SEQ ID NO: 5;
a TRIM55-specific primer pair comprises TRIM55-F having the nucleotide sequence of SEQ ID NO: 6 and TRIM55-R having the nucleotide sequence of SEQ ID NO: 7; and
a GAS7-specific primer pair comprises GAS7-F having the nucleotide sequence of SEQ ID NO: 8 and GAS7-R having the nucleotide sequence of SEQ ID NO: 9.

18. A method for genotyping a pig economic trait gene, comprising applying the kit according to claim 3.

19. The method according to claim 18, wherein the restriction endonuclease is selected from the group consisting of ScaI-HF, BstUI, and HindIII.


SEQUENCE LISTING (16 sequences)

SEQ ID NO: 1 - DNA, 363 bp, synthetic construct
  source 1..363: mol_type = other DNA; note = WIP1 gene
aggaatcaaccctgcttacatctggatttcaaacttctggcctccaaaatcatgagagaacaaatttccgttgttttaagccacccaatttctgctaatttgtgacagcacctctacaaaacaaatacagcaattctgttcctcaaattttttacataactagttttgtaacatttatgatgtttcttggctgaacaattgattatgatggttatcaaatgtgaatttttagttccatcattccttctacattcatcagcggacttttataaagtaatgatgattaacaaaaatggggaaaaacaagtacktgtcaagtagggcaaattcgttcagaacatatgatgtgtgagagggtgaagg

SEQ ID NO: 2 - DNA, 247 bp, synthetic construct
  source 1..247: mol_type = other DNA; note = TRIM55 gene
acaagagtgtagtgccgacttttttcaacacttgccacttttactgacrcgcacacacacacataccacacctacacaccctttgtaatgtttcccacacgataaagatttgcataagatcaagttcccctttttttcctaatgccttctgcaactattttctcaaagcatggttttccctcactgtgttagataagtcatctggaattaaaaggtatcttttatagaaatcctgttcagaggtccc

SEQ ID NO: 3 - DNA, 298 bp, synthetic construct
  source 1..298: mol_type = other DNA; note = GAS7 gene
gaagccagttaacaggaaacytgccactccctgggagacgtcagccactttccaaccacctccctccagccttcccccttggggtccagccctccagaaacaacagaccgtcctcctcccccctcccccacaagccaggagaaggccaagggcacgggactctgcgaggatggatcacgccagcggcaggtctgcgccggatccctctccaccctgcccgcctctcacgccggccctcatttgcaggagacgtgccgctgtagctgagtgtatatatcgtctggatgatttgccgtgg

SEQ ID NO: 4 - DNA, 18 bp, synthetic construct
  source 1..18: mol_type = other DNA; note = Primer WIP1-F
aggaatcaaccctgctta

SEQ ID NO: 5 - DNA, 18 bp, synthetic construct
  source 1..18: mol_type = other DNA; note = Primer WIP1-R
ccttcaccctctcacaca

SEQ ID NO: 6 - DNA, 20 bp, synthetic construct
  source 1..20: mol_type = other DNA; note = Primer TRIM55-F
gggacctctgaacaggattt

SEQ ID NO: 7 - DNA, 21 bp, synthetic construct
  source 1..21: mol_type = other DNA; note = Primer TRIM55-R
acaagagtgtagtgccgactt

SEQ ID NO: 8 - DNA, 20 bp, synthetic construct
  source 1..20: mol_type = other DNA; note = Primer GAS7-F
gaagccagttaacaggaagc

SEQ ID NO: 9 - DNA, 17 bp, synthetic construct
  source 1..17: mol_type = other DNA; note = Primer GAS7-R
ccacggcaaatcatcca

SEQ ID NO: 10 - DNA, 20 bp, synthetic construct
  source 1..20: mol_type = other DNA; note = Original primer sequence for GAS7
gaagccagttaacaggaaac

SEQ ID NO: 11 - DNA, 13 bp, synthetic construct
  source 1..13: mol_type = other DNA; note = Sequencing result showing the GG genotype of WIP1
aagtacgtgtcaa

SEQ ID NO: 12 - DNA, 13 bp, synthetic construct
  source 1..13: mol_type = other DNA; note = Sequencing result showing the TG genotype of WIP1
aagtacktgtcaa

SEQ ID NO: 13 - DNA, 13 bp, synthetic construct
  source 1..13: mol_type = other DNA; note = Sequencing result showing the AG genotype of TRIM55
actgacrcgcacg

SEQ ID NO: 14 - DNA, 13 bp, synthetic construct
  source 1..13: mol_type = other DNA; note = Sequencing result showing the AA genotype of TRIM55
actgacacgcacg

SEQ ID NO: 15 - DNA, 13 bp, synthetic construct
  source 1..13: mol_type = other DNA; note = Sequencing result showing the TT genotype of GAS7
ggaagcctgccac

SEQ ID NO: 16 - DNA, 13 bp, synthetic construct
  source 1..13: mol_type = other DNA; note = Sequencing result showing the TC genotype of GAS7
ggaagcytgccac